(19) United States
(12) Patent Application Publication
(10) Pub. No.: US 2026/0272688 A1
(43) Pub. Date: Sep. 17, 2026

(54) DELIVERY SLEEVES FOR MULTI BRANCH EXPANDABLE MEDICAL DEVICES

(71) Applicant: W. L. Gore & Associates, Inc., Newark, DE (US)

(72) Inventors: Merrill J. Birdno, Flagstaff, AZ (US); Jerald M. Crawley, Flagstaff, AZ (US); Logan R. Hagaman, Newark, DE (US); James A. McMaster, Newark, DE (US)

(21) Appl. No.: 19/565,756

(22) Filed: Mar. 13, 2026

Related U.S. Application Data
(60) Provisional application No. 63/772,021, filed on Mar. 14, 2025.

(51) Int. Cl. A61F 2/97; A61F 2/07; A61F 2/954

(52) U.S. Cl. CPC A61F 2/97; A61F 2/07; A61F 2/954

(57) ABSTRACT

A deployment system for a branched endoluminal device of the type including one or more openings and one or more sleeves having a delivery configuration circumferentially wrapped around at least portions of the endoluminal device. Each of the sleeves defines a seamline substantially opposite the one or more openings, and the sleeves are attached to the endoluminal device at a plurality of attachment locations circumferentially located sufficiently close to the sleeve seamline to prevent the sleeve from substantially covering any of the one or more openings after the sleeve is released and the endoluminal device has expanded to a deployed diameter configuration.

### CROSS-REFERENCE TO RELATED APPLICATION

This application claims the benefit of Provisional Application No. 63/772,021, filed Mar. 14, 2025, which is incorporated herein by reference in its entirety for all purposes.

### FIELD

The present disclosure relates generally to implantable endoluminal devices and related deployment systems and methods. More specifically, the disclosure relates to branched endoluminal devices with one or more openings on their sides, and related deployment systems and methods.

### BACKGROUND

A variety of branched, anatomical passages may benefit from treatment in the form of an implanted, endoluminal device. One such passage is a vascular passage, such as an artery, with an aneurysm. Aortic disease and trauma such as aneurysms and dissections may present significant risk to a patient. That risk may be increased based on the patient's condition. Such conditions or factors can include the patient's age and preexisting and/or related conditions such as cardiopulmonary bypass, cardiac arrest, circulatory arrest. These and other factors may limit the patient's ability to withstand and recover from surgery to repair the aortic disease. This same issue may exist in other diseased and damaged tissues in patients.

With respect to aneurysms, in order to prevent rupturing of an aneurysm, a stent graft may be introduced into a blood vessel percutaneously, and deployed to span the aneurysmal sac. Stent grafts include a graft secured to a cylindrical scaffolding or framework of one or more stents. The stent(s) provide rigidity and structure to hold the graft open in a tubular configuration as well as the outward radial force needed to create a seal between the graft and a healthy portion of the vessel wall and provide migration resistance. Blood flowing through the vessel can be channeled through the luminal surface of the stent graft to reduce, if not eliminate, the stress on the vessel wall at the location of the aneurysmal sac. Stent grafts may reduce the risk of rupture of the blood vessel wall at the aneurysmal site and allow blood to flow through the vessel without interruption.

Various endovascular repair procedures such as the exclusion of an aneurysm require a stent graft to be implanted adjacent to a vascular bifurcation. Often the aneurysm extends into the bifurcation requiring the stent graft to be placed into the bifurcation. A bifurcated stent graft that, for example, includes one or more openings such as side branch portals, may be used in these cases. Modular stent grafts, having a separate main body and branch components, may be preferred in these procedures due factors such as the ease and accuracy of deployment. U.S.

Patent Application Publication No. 2008/0114446 to Hartley et al. is an example of a modular stent graft having separate main body and branch stent components. In the Hartley et al. publication the main body stent has a fenestration in the side wall that is tailored to engage and secure the side branch stent.

Endoluminal devices, including such stent graft devices with one or more side branch portals are typically delivered via a catheter assembly. Stent grafts may have a collapsed, reduced diameter delivery configuration for delivery to the patient's vasculature, and then be radially expanded toward an increased diameter deployed configuration after the treatment area of the vasculature is reached. Stent grafts may be constrained in the collapsed configuration by a biocompatible sleeve.

### SUMMARY

Disclosed embodiments include deployment systems for use in connection with endoluminal devices having one or more side branch portals. The deployment systems include sleeves that may be substantially clear of the side branch portals and/or one or more of the openings on the proximal and distal ends of the devices following deployment. Advantages of these types may be provided without excessive or substantial sleeve overhang upon deployment. They may maintain head vessel patency and head vessel pressures. They may also maintain the ability to effectively and efficaciously cannulate the branch vessels. The devices may also be efficiently manufactured.

According to one example (“Example 1”), a deployment system for a branched endoluminal device is provided. The deployment system includes an expandable endoluminal device having an exterior surface and an interior surface defining a sidewall, and including at least a first portion and a second portion expandable from a collapsed delivery diameter configuration toward deployed diameter configurations. The second portion includes one or more openings through the sidewall. The deployment system includes a first sleeve including first and second edge portions. The first sleeve has a delivery configuration circumferentially wrapped around at least portions of the first and second portions of the endoluminal device with the first and second edge portions releasably coupled to define a first sleeve seamline substantially opposite the one or more openings. The first sleeve is attached to the endoluminal device at a plurality of attachment locations circumferentially located sufficiently close to the first sleeve seamline to prevent the first sleeve from substantially covering any of the one or more openings after the first and second edge portions are released and the first and second portions of the endoluminal device have expanded to the deployed diameter configurations. The deployment system includes a second sleeve including first and second edge portions. The second sleeve has a delivery configuration circumferentially wrapped around the first portion of the endoluminal device with the first and second edge portions releasably coupled to define a second sleeve seamline adjacent to the first sleeve seamline. The second sleeve is attached to the endoluminal device at a plurality of attachment locations circumferentially located sufficiently close to the first seamline to prevent the second sleeve from substantially covering any of the one or more openings after the first and second edge portions are released and the second portion of the endoluminal device has expanded to the deployed diameter configuration.

According to another example (“Example 2”), further to Example 1, the first sleeve is attached to the endoluminal device at attachment locations including first locations within about 30° from the first sleeve seamline.

According to another example (“Example 3”), further to Example 1, the first sleeve is attached to the endoluminal device at attachment locations including first locations within about 15° from the first sleeve seamline.

According to another example (“Example 4”), further to Example 1, the first locations include at least one location on each of the first portion and the second portion of the endoluminal device.

According to another example (“Example 5”), further to Example 4, the first locations include at least one location on the first portion of the endoluminal device, and at least two locations on the second portion of the endoluminal device.

According to another example (“Example 6”), further to Example 5, at least one of the first locations is a location attaching both the first sleeve and the second sleeve to the endoluminal device.

According to another example (“Example 7”), further to Example 5, the endoluminal device includes a proximal end on the first portion, opposite the first portion from the second portion. The first sleeve and the second sleeve are attached to the endoluminal device sufficiently close to the first sleeve seamline to prevent the first sleeve and the second sleeve from substantially covering the proximal end of the endoluminal device after the first and second portions of the endoluminal device have expanded to the deployed diameter.

According to another example (“Example 8”), further to Example 7, the first sleeve and the second sleeve are attached to the endoluminal device at attachment locations including second locations between the first locations and the proximal end of the endoluminal device. The second locations are locations between about 30° and about 65° from the first seamline.

According to another example (“Example 9”), further to Example 8, the second locations are locations between about 50° and about 60° from the first seamline.

According to another example (“Example 10”), further to Example 8, the second locations are on a proximal end portion of the endoluminal device.

According to another example (“Example 11”), further to Example 8, the second locations are the same locations.

According to another example (“Example 12”), further to Example 1, the endoluminal device includes a third portion opposite the second portion from the first portion, and the third portion is expandable from a collapsed delivery diameter configuration toward a deployed diameter configuration. The first sleeve, in the delivery configuration, circumferentially wraps around at least a portion of the third portion of the endoluminal device. The first sleeve is attached to the endoluminal device at a plurality of attachment locations circumferentially located sufficiently close to the first sleeve seamline to prevent the first sleeve from substantially covering any of the one or more openings after the first, second and third portions of the endoluminal device have expanded to the deployed diameter configurations. The deployment system includes a third sleeve including first and second edge portions. The third sleeve has a delivery configuration circumferentially wrapped around the third portion of the endoluminal device with the first and second edge portions releasably coupled to device to define a third seamline. The third sleeve is attached to the endoluminal device at a plurality of attachment locations circumferentially located sufficiently close to the first seamline to prevent the third sleeve from substantially covering any of the one or more openings after the first and second edge portions are released and the third portion of the endoluminal device has expanded to the deployed diameter configuration.

According to another example (“Example 13”), further to Example 12, the first sleeve is attached to the endoluminal device at attachment locations including first locations within about 30° from the first sleeve seamline.

According to another example (“Example 14”), further to Example 12, the first locations include at least one location on each of the second portion and the third portion of the endoluminal device.

According to another example (“Example 15”), further to Example 14, at least one of the first locations is a location attaching both the first sleeve and the third sleeve to the endoluminal device.

According to another example (“Example 16”), further to Example 14, the endoluminal device includes a distal end on the third portion, opposite the third portion from the second portion. The first sleeve and the third sleeve are attached to the endoluminal device sufficiently close to the first sleeve seamline to prevent the first sleeve and the third sleeve from substantially covering the distal end of the endoluminal device after the first and third portions of the endoluminal device have expanded to the deployed diameter.

According to another example (“Example 17”), further to Example 16, the first sleeve and the third sleeve are attached to the endoluminal device at attachment locations including second locations between the first locations and the distal end of the endoluminal device. The second locations are locations between about 30° and about 50° from the first seamline.

According to another example (“Example 18”), further to Example 17, the second locations are locations between about 35° and about 45° from the first seamline.

According to another example (“Example 19”), further to Example 17, the second locations are on a distal end portion of the endoluminal device.

According to another example (“Example 20”), further to Example 17, the second locations are the same locations.

According to another example (“Example 21”), further to any of Examples 1-20, a method for treating a patient using the deployment system is provided. The method includes delivering the deployment system to a branched vessel including one or more branches. The method includes locating the deployment system in the branched vessel, and positioning the deployment system with the one or more openings of the endoluminal device facing the one or more branches of the vessel, and at least the first and second seamlines facing a side of the branched vessel opposite the one or more branches of the vessel. The method includes releasing the first and second edge portions of the first sleeve to cause the second portion of the endoluminal device to expand to its deployed configuration and to cause the first portion of the endoluminal device to expand to its intermediate configuration. Following the expansion of the second portion of the endoluminal device, the first sleeve does not substantially cover any of the one or more openings through the sidewall of the endoluminal device.

According to another example (“Example 22”), further to Example 21, the method includes releasing the first and second edge portions of the second sleeve to cause the first portion of the endoluminal device to expand to its deployed configuration. Following the expansion of the first portion of the endoluminal device to its deployed configuration, the second sleeve does not substantially cover any of the one or more openings through the sidewall of the endoluminal device.

According to another example (“Example 23”), further to Example 22, the method includes repositioning the deployment system after the expansion of the second portion of the endoluminal device, and before causing the expansion of the first portion of the endoluminal device.

According to one example (“Example 24”), a deployment system for a branched endoluminal device is provided. The system includes an expandable endoluminal device including at least a first portion, a third portion, and second portion between the first portion and the third portion. The first, second and third portions are expandable toward deployed diameter configurations. The second portion includes one or more side branch portals. The system includes a first sleeve including a first edge portion and a second edge portion wrapped circumferentially about the first, second and third portions of the endoluminal device. The first edge portion and the second edge portion are releasably coupled via a first coupling member to define a first sleeve seamline substantially opposite the endoluminal device from the one or more side branch portals and to maintain the first, second and third portions of the endoluminal device in collapsed delivery diameter configurations that are less than the deployed diameter configurations. The system includes a first sleeve attachment attaching the first sleeve to the endoluminal device at a plurality of first sleeve attachment locations circumferentially located within about 30° from the first seamline. The system includes a second sleeve including a first edge portion and a second edge portion wrapped circumferentially about the first portion of the endoluminal device. The first edge portion and the second edge portion are releasably coupled via a second coupling member to define a second sleeve seamline adjacent to the first sleeve seamline and to maintain the first portion of the endoluminal device in a collapsed intermediate diameter configuration that is less than the deployed diameter configuration of the second portion. The system includes a second sleeve attachment attaching the second sleeve to the endoluminal device at a plurality of second sleeve attachment locations circumferentially located within about 65° from the second seamline. The system includes a third sleeve including a first edge portion and a second edge portion wrapped circumferentially about the third portion of the endoluminal device. The first edge portion and the second edge portion are releasably coupled via a third coupling member to define a third sleeve seamline adjacent to the first sleeve seamline and to maintain the third portion of the endoluminal device in a collapsed intermediate diameter configuration that is less than the deployed diameter configuration of the third portion. The system includes a third sleeve attachment attaching the third sleeve to the endoluminal device at a plurality of third sleeve attachment locations circumferentially located within about 50° from the first seamline.

According to another example (“Example 25”), further to Example 24, the first sleeve attachment locations include at least three attachment locations.

According to another example (“Example 26”), further to Example 25, the first sleeve attachment locations are located within about 15° from the first sleeve seamline.

According to another example (“Example 27”), further to Example 25, the first sleeve attachment locations include at least one location on each of the first, second and third portions of the endoluminal device.

According to another example (“Example 28”), further to Example 27, the first sleeve attachment locations include at least one location on the first portion of the endoluminal device, and at least two locations on the second portion of the endoluminal device.

According to another example (“Example 29”), further to Example 28, at least one of the first sleeve attachment locations is a location attaching both the first sleeve and the second sleeve to the endoluminal device.

According to another example (“Example 30”), further to Example 29, the endoluminal device includes a proximal end on the first portion, opposite the first portion from the second portion. The first sleeve and the second sleeve are attached to the endoluminal device at attachment locations including second locations between the first attachment locations and the proximal end of the endoluminal device. The second locations are locations between about 30° and about 65° from the first seamline.

According to another example (“Example 31”), further to Example 30, the second locations are locations between about 50° and about 60° from the first seamline.

According to another example (“Example 32”), further to Example 31, the second locations are on a proximal end portion of the endoluminal device.

According to another example (“Example 33”), further to Example 31, the second locations are the same locations.

According to another example (“Example 34”), further to Example 24, the first sleeve attachment locations include at least one location on each of the second portion and the third portion of the endoluminal device.

According to another example (“Example 35”), further to Example 34, at least one of the first sleeve attachment locations is a location attaching both the first sleeve and the third sleeve to the endoluminal device.

According to another example (“Example 36”), further to Example 34, the endoluminal device includes a distal end on the third portion, opposite the third portion from the second portion. The first sleeve and the third sleeve are attached to the endoluminal device at attachment locations including third locations between the first attachment locations and the distal end of the endoluminal device. The third locations are locations between about 30° and about 50° from the first seamline.

According to another example (“Example 37”), further to Example 36, the third locations are locations between about 35° and about 45° from the first seamline.

According to another example (“Example 38”), further to Example 36, the third locations are on a distal end portion of the endoluminal device.

According to another example (“Example 39”), further to Example 36, the third locations are the same locations.

According to another example (“Example 40”), further to any of Examples 24-39, a method for treating a patient using the deployment system is provided. The method includes delivering the deployment system to a branched vessel including one or more branches. The method includes locating the deployment system in the branched vessel, and positioning the deployment system with the one or more side branch portals of the endoluminal device facing the one or more branches of the vessel, and the first, second and third seamlines facing a side of the branched vessel opposite the one or more branches of the vessel. The method includes releasing the first and second edge portions of the first sleeve to cause the second portion of the endoluminal device to expand to its deployed configuration and to cause the first and second portions of the endoluminal device to expand to their intermediate configurations. After causing the second portion of the endoluminal device to expand to its deployed configuration, the method includes releasing the first and second edge portions of the second sleeve to cause the first portion of the endoluminal device to expand to its deployed configuration. After causing the second portion of the endoluminal device to expand to its delivery configuration, the method includes releasing the first and second edge portions of the third sleeve to cause the third portion of the endoluminal device to expand to its deployed configuration.

According to another example (“Example 41”), further to Example 40, the method includes repositioning the deployment system after causing the second portion of the endoluminal device to expand to its deployed configuration, and before one or both of causing the first portion of the endoluminal device to expand or causing the third portion of the endoluminal device to expand.

According to another example (“Example 42”), further to Example 40, the method includes simultaneously releasing the first and second edge portions of the second sleeve and the first and second edge portions of the third sleeve.

According to one example (“Example 43”), a method for treating vessel of a patient including one or more branches with a deployment system including a branched endoluminal device is provided. The endoluminal device includes a first portion that is expandable to a first portion deployed configuration, and at least a second portion including one or more side branch openings that is expandable to a second portion deployed configuration. The method includes delivering the deployment system to the branched vessel while the first and second portions of the endoluminal device are constrained by a first sleeve to a delivery configuration and the first portion is constrained by a second sleeve to an intermediate deployed configuration, and positioning the deployment system with the one or more side branch openings of the endoluminal device facing the one or more branches of the vessel. The method includes actuating the deployment system to release the first sleeve, to cause the second portion of the endoluminal device to expand to its second portion deployed configuration, and to cause the first portion of the endoluminal device to expand to its intermediate deployed configuration.

Following the release of the first sleeve the first sleeve does not substantially cover any of the side branch openings of the endoluminal device. After actuating the deployment system to release the first sleeve, the method includes actuating the deployment system to release the second sleeve, to cause the first portion of the endoluminal device to expand to its first portion deployed configuration. Following the release of the second sleeve the second sleeve does not substantially cover any of the side branch openings of the endoluminal device.

According to another example (“Example 44”), further to Example 43, the endoluminal device includes a third portion opposite the second portion from the first portion and that is expandable to a third portion deployed configuration.

Delivering and positioning the deployment system while the first and second portions of the endoluminal device are constrained by the first sleeve to the delivery configuration includes delivering the deployment system to the branched vessel while the third portion is constrained by the first sleeve to the delivery configuration and the third portion is constrained by a third sleeve to an intermediate deployed configuration. Actuating the deployment system to release the first sleeve includes causing the third portion of the endoluminal device to expand to its intermediate deployed configuration. After actuating the deployment system to release the first sleeve, the method includes actuating the deployment system to release the third sleeve, to cause the third portion of the endoluminal device to expand to its third portion deployed configuration. Following the release of the third sleeve the third sleeve does not substantially cover any of the side branch openings of the endoluminal device.

According to another example (“Example 45”), further to Example 44, the method includes repositioning the deployment system after actuating the deployment system to release the first sleeve, and before actuating the deployment system to release one or both of the first sleeve or the second sleeve. The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a diagrammatic illustration of a branched endoluminal device in an aortic arch of a patient.

FIG. 2 is a top view of a deployment system including a branched endoluminal device and sleeves in an unconstrained configuration, in accordance with embodiments.

FIG. 3 is a side view of the deployment system in a delivery configuration, in accordance with embodiments.

FIG. 4 is an isometric view of the deployment system in a delivery configuration, in accordance with embodiments.

FIG. 5A is a sectional view of the deployment system, taken along lines A-A in FIG. 4, in accordance with embodiments.

FIG. 5B is a sectional view of the deployment system, taken along lines B-B in FIG. 4, in accordance with embodiments.

FIG. 6 is an illustration of the deployment system in an opened, flat configuration, in accordance with embodiments.

FIG. 7 is a detailed illustration of a portion or an endoluminal device, including side branch portals, in accordance with embodiments.

FIG. 8 is a diagrammatic illustration of a delivery device, in accordance with embodiments.

FIGS. 9A-9C are illustrations of portions of a deployment system and delivery device, in accordance with embodiments.

### DETAILED DESCRIPTION

### Definitions and Terminology

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Certain relative terminology is used to indicate the relative position of components and features. For example, words such as “top”, “bottom”, “upper,” “lower,” “left,” “right,” “horizontal,” “vertical,” “upward,” “downward,” “distal,” “proximal,” “leading” and “trailing” are used in a relational sense (e.g., how components or features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

With respect to terminology of inexactitude, the terms “about” and “approximately” may be used, in certain instances, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Similarly, with term “substantially” may be used, in certain instances, to refer to an objective, result or outcome that includes the stated objective, result or outcome, and that also includes objectives, results or outcomes that are reasonably close to the stated objective, result or outcome. Objectives, results or outcomes that are reasonably close to the stated objective, result or outcome may deviate from the stated objective, result or outcome by a reasonably small amount as understood and readily ascertained by individuals having ordinary skill in the relevant arts. For example, objectives, results or outcomes that provide medically efficacious results may substantially meet the stated objective, result or outcome.

As used herein, “couple” means join, connect, attach, adhere, affix, or bond, whether directly or indirectly, and whether permanently or temporarily.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is considered not an elastomer or elastomeric material as is generally known.

The term “film” as used herein generically refers to one or more of the membrane, composite material, or laminate.

The term “biocompatible material” as used herein generically refers to any material with biocompatible characteristics including synthetic materials, such as, but not limited to, a biocompatible polymer, or a biological material, such as, but not limited to, bovine pericardium. Biocompatible material may comprise a first film and a second film as described herein for various embodiments.

For reference, the terms “circumference” “circumferentially” and “diameter” are not meant to require a circular cross-section or path (although are inclusive of a circular cross-section or path), and are instead to be understood broadly to reference an outer surface or dimension, the dimension between opposing sides of the outer surface, and paths or directions along such outer surfaces or dimensions.

Although the embodiments herein may be described in connection with various principles and beliefs, the described embodiments should not be bound by theory. For example, embodiments are described herein in connection with vascular stent grafts, and more specifically branched stent grafts. However, embodiments within the scope of this disclosure can be applied toward any endoprostheses of similar structure and/or function. Furthermore, embodiments within the scope of this disclosure can be applied in non-vascular applications.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various embodiments relate to stent graft and associated sleeve enhancements. Some aspects facilitate device positioning, sleeve release and device expansion, an ability to orient device branches and avoidance of branch opening interference. These, or additional or alternative advantages and features will be understood with reference to the description that follows and the associated drawings.

The endoluminal devices and delivery systems shown herein are provided as an examples of the various features of the endoluminal devices and delivery systems, and although the combination of those illustrated features is clearly within the scope of invention, those examples and illustrations are not meant to suggest the inventive concepts provided herein are limited from fewer features, additional features, or alternative features to one or more of those features shown. For example, in various embodiments, the endoluminal device may include more or fewer side branch portals, and the sleeves can be on opposite sides of the endoluminal device. The various portions of the endoluminal device may have different diameters when in their expanded states, deployed in a patient. Certain sizes and dimensions of features may vary depending on the size of the vessels at the treatment site for a given patient (e.g., different patients have different sized vessels). It should also be understood that the reverse is true as well. One or more of the components depicted in the illustrated examples can be employed in addition to, or as an alternative to the components depicted.

Devices, systems, and methods in accordance with various embodiments are disclosed herein for treating disease of human vasculature.

Although the description below and figures are illustrated in the context of treating the aorta, including the ascending aorta, aortic arch, and descending aorta, and branches therefrom, including for example the brachiocephalic artery, the left common carotid artery, and the left subclavian artery, it should be appreciated that the present disclosure can be applied to treatment of other portions of the vasculature or, including, for example, any disease where a larger vessel and one or more branch vessels are to be treated.

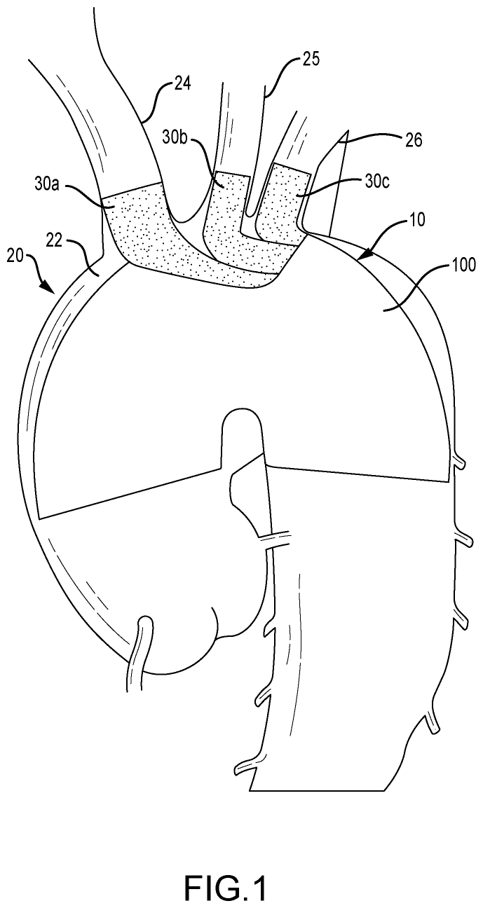

Referring to FIG. 1, an implantable endoluminal device 10 including a main body 100 can be delivered and deployed in the aorta 20. Branch bodies 30 can be deployed in branching arteries (e.g., a first branch body 30a in the brachiocephalic artery (BCA) 24, a second branch body 30b in the left common carotid (LCCA) artery 25, and a third branch body 30c in the left subclavian artery (LSA) 26. The branch bodies 30 extend from one or more side branch portals in the main body 100.

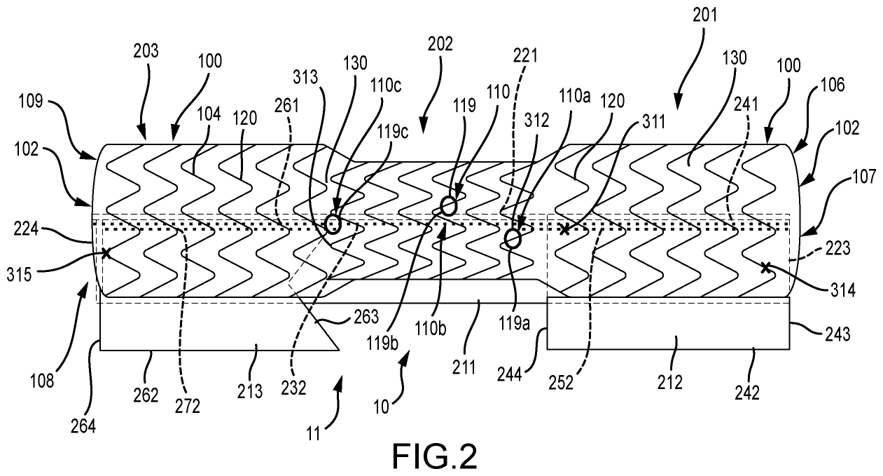

FIG. 2 is an exemplary embodiment of a deployment system 11 including an implantable device 10 and deployment sleeves 211, 212 and 213. The main body 100 includes a wall 104 forming the main lumen 102. The main body 100 has a proximal or first end 106 and a distal or second end 108. At the first end 106 the main body 100 includes a first opening 107 into the lumen 102, and at the second end 108 the main body 100 includes a second opening 109 into the lumen.

Each of the openings 107, 109 provides access to the main lumen 102 at the corresponding end 106, 108. Fluids are operable to flow through the main lumen 102 by passing through the first opening 107, into the main lumen 102, and out the second opening 109, defining a main body fluid flow direction. Or, the flow may be in the opposite direction, defining the main body fluid flow direction. The outer wall 104 substantially forms or defines the outer profile of the main body 100. In embodiments, the first end 106 of the main body 100 can be referred to as a proximal or leading end of the implantable device 10, and may be the end located closest to a heart of the patient following deployment in an aorta. Following this convention, the second end 108 of the main body 100 can be referred to as a distal or trailing end of the implantable device 10, and may be the end located furthest from the heart of the patient following deployment in the aorta.

In some embodiments, the main body 100 is formed of a stent structure 120 and a graft member 130. The stent structure 120 is operable to maintain patency of the main body 100 and/or the main vessel (e.g., the aorta) when the main body 100 is deployed. The stent structure 120 can be formed of various materials, including, but not limited to, metals, metal alloys, polymers, and any combination thereof to provide elastic or plastic properties (e.g., self-expanding or balloon-expandable stents). The graft member 130 is coupled to the stent structure 120 and forms the fluid impermeable or semi-permeable layer through which fluids may flow (e.g., blood). In the illustrated embodiments, the stent structure 120 includes lengths of undulating elongated elements, defining apices.

Nitinol (NiTi) may be used as the material of the stent structure 120, but other materials such as, but not limited to, stainless steel, L605 steel, polymers, MP35N steel, polymeric materials, Pyhnox, Elgiloy, or any other appropriate biocompatible material, and combinations thereof, can be used as the material of the stent structure. The super-elastic properties and softness of NiTi may enhance the conformability of the stent structure 120. In addition, NiTi can be shape-set into a desired shape. That is, NiTi can be shape-set so that the stent structure 120 tends to self-expand into a desired shape when the frame is unconstrained, such as when the stent structure is deployed out from a delivery system.

Potential materials for the graft member 130 include, for example, expanded polytetrafluoroethylene (ePTFE), polyester, polyurethane, fluoropolymers, such as perfluoroelastomers and the like, polytetrafluoroethylene, silicones, urethanes, ultra-high molecular weight polyethylene, expanded polyethylene (ePE), aramid fibers, and combinations thereof. Other embodiments for a graft member 130 material can include high strength polymer fibers such as ultra-high molecular weight polyethylene fibers (e.g., Spectra®, Dyneema Purity®, etc.) or aramid fibers (e.g., Technora®, etc.). The graft member 130 may include a bioactive agent. In one embodiment, an ePTFE graft includes a carbon component along a blood contacting surface thereof. Any graft member which can be delivered by a catheter is in accordance with the present disclosure.

The main body 100 further includes at least one side branch portal 110. The side branch portal 110 is operable to provide fluidic access between the main lumen 102 and a branch vessel. The side branch portal 110 forms or is positioned in an opening 119 through the wall 104 along the outer profile of the main body 100. In certain instances, the side branch portal 110 extends through the wall 104 of the main body 100 longitudinally between the first end 106 and the second end 108 of the main body 100. Thus, fluid may flow through the first opening 107 and through the side branch portal 110. Some embodiments include a plurality of side branch portals 110. For example, FIG. 2 illustrates a main body 100 included a first side branch portal 110a, a second side branch portal 110b, and a third side branch portal 110c. Any number of side branch portals 110 may be incorporated to accommodate the specific anatomy into which the device 10 is to be deployed.

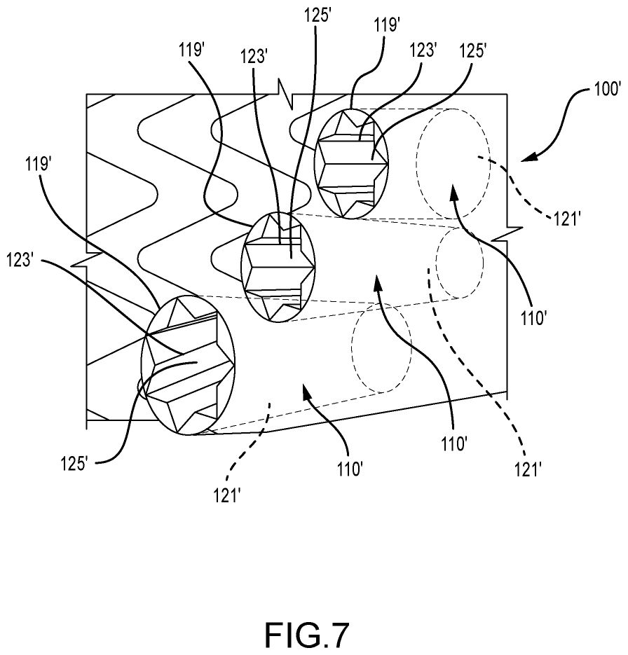

In some embodiments (e.g., as shown in FIG. 1), each of the side branch portals 110 includes a side branch stent structure and a side branch graft member. In various embodiments, the side branch stent structure and side branch graft member can be independent from, incorporated into, or integral with the main body stent structure 120 and the main body graft member 130. For example, the side branch stent structures may be separate or independent from the main body stent structure 120, whereas the side branch graft member is incorporated into the main body graft member 130 (e.g., sandwiched or interposed between layers of the main body graft member 130). In some embodiments, the side branch stent structure extends from the main body stent structure 120 and therefore represents a portion of the main body stent structure 120 rather than an independent stent structure. In still other embodiments, the side branch stent structure is coupled to the main body stent structure 120. Similarly, the side branch graft member can be formed directly from the main body graft member 130 and therefore represent a portion of the main body graft member 130. In other embodiments, the side branch graft member is coupled to the main body graft member 130 or, or in still other embodiments, is spaced from the main body graft member 130. It is understood that any combination of side branch stent structures and side branch graft member embodiments is within the scope of this disclosure. FIG. 7, for example, illustrates a portion of a main body 100′ that includes side branch portals 110′ and openings 119′ in accordance with embodiments. The side branch portals 110′ include tubular portions 121′ extending into the lumen of the main body 100′ from the openings 119′. In some embodiments such as those shown in FIG. 7, the side branch portals 110′ may include a side branch stent structure 123′ and/or a side branch graft member 125′. Side branch portals, including side branch stent structures and side branch graft members of these types are disclosed, for example, in the Birdno U.S. Patent Application Publication No. 2024/0130877.

In some embodiments, the side branch portal 110 is positioned between the first end 106 and the second end 108 of the main body 100 and does not extend beyond or increase the outer profile of the main body 100. Stated otherwise, the portion of an outer wall of the side branch portal 110 is positioned along the wall 104 of the main body 100 within the outer profile of the main body 100 (e.g., flush with the outer profile). Thus, the side branch portal 110 may extend into the main lumen 102 of the main body 100 without substantially increasing the outer profile of the main body 100 adjacent the exit location such as the opening 119 of the side branch portal 110 from the main body 100.

Each side branch portal 110 includes an end defining an opening 119. Fluids travel from the main lumen 102 through each side branch portal 110 to the opening 119 (or vice versa) defining a side branch fluid flow direction. The side branch portal 110 is positioned such that the opening 119 is positioned exterior to or oriented away from the main body 100 (e.g., the opening 119 is an exterior opening of the side branch portal 110 relative to the wall 104 and main lumen 102 of the main body 100). In embodiments, for example, a lumen defining the opening 119 of the side branch portal 110 extends to and may be positioned within the main lumen 102. The side branch portal 110 may have various longitudinal lengths. Furthermore, when a plurality of side branch portals 110 are implemented, each side branch portal 110 may include various lengths or may be uniform in length. It is understood that in embodiments implementing a plurality of side branch portals 110, each side branch portal 110 may have an independent diameter or geometric orifice area.

In some embodiments, the side branch portal 110 is oriented such that the side branch fluid flow direction is opposite to the main body fluid flow direction (e.g., retrograde to the main body fluid flow direction). It is understood that opposite or retrograde in these embodiments is not limited to 180 degrees of difference, but generally encompasses a change in the direction of the fluid flowing that is greater than 90 degrees. It is also understood that the direction of the fluid flow is with respect to the specific location along the longitudinal length of the main body 100 as the main body may conform to a curved anatomy. By orienting the side branch portal 110 in the retrograde orientation, a surgeon may be able to perform the intervention and any subsequent interventions from a more advantageous access site (e.g., femoral access site to reduce trauma to carotid arteries, subclavian, or other arteries or decrease surgical presence in more anatomically crowded portions of a patient such as around the neck or thorax when operating in the aortic arch).

This orientation may be advantageous in some presentations where access may difficult, obstructed, or dangerous from certain access sites.

In other embodiments, the side branch portal 110 is oriented such that the side branch fluid flow direction is generally oriented with the main body fluid flow direction (e.g., antegrade to the main body fluid flow direction). Antegrade orientations may be advantageous in some embodiments to maintain more traditional fluid flow, especially in tissues or anatomies that may have unique geometries that would limit the use of a retrograde orientation. In embodiments implementing a plurality of side branch portals 110, the side branch portals may all have an antegrade orientation, may all have a retrograde orientation, or may include one or more branch portals with an antegrade orientation and one or more portals having a retrograde orientation.

The opening 119 of the side branch portal 110 can be positioned at various longitudinal positions between the first end 106 and the second end 108 of the main body 100. For example, the opening 119 of the side branch portal 110 may be positioned generally at the midpoint between the first and second ends 106, 108 of the main body 100. In other embodiments, the opening 119 of the side branch portal 110 may be positioned closer to the first end 106 relative the second end 108 or, alternatively, closer to the second end 108 relative to the first end 106 of the main body 100. In those embodiments including a plurality of side branch portals 110, each opening 119 may be aligned longitudinally along the length of the main body 100, staggered or offset along the length of the main body 100, or a combination thereof. In the embodiments shown in FIG. 2, for example, the opening 119a of side branch portal 110 is located closer to the first end 106 of the main body 100 than the location of the opening 119b of the side branch portal 110b. In these embodiments the opening 119c of the side branch portal 110c is located closer to the second end 108 of the main body 100 than the location of the opening 119b of the side branch portal 110b. The longitudinally staggered locations of the openings 119a-119c of the embodiments shown in FIG. 2 can, for example, correspond to locations of the BCA 24, LCCA 25 and LSA 26 in a patient.

The opening 119 of the side branch portal 110 can also be positioned at various circumferential positions around the main body 100. In those embodiments including a plurality of side branch portals 110, for example, the openings 119 of the side branch portals 110 may be aligned along a circumference of the main body 100 (e.g., a circumference defining a plane perpendicular to a longitudinal axis of the main body), staggered along a circumference of the main body, or a combination thereof. In the embodiments shown in FIG. 2, for example, the opening 119a of the first side branch portal 110a and the opening 119c of the third side branch portal 110c are at locations circumferentially staggered or offset with respect to the circumferential location of the opening 119b of the second side branch portal 110b. In these embodiments the circumferential location of the opening 119a of the first side branch portal 110a is circumferentially staggered or offset from the location of the opening 119b of the second side branch portal 110b by a distance that is greater than the distance that the opening 119c of the third side branch portal 110c is circumferentially staggered or offset from the location of the opening 119b of the second side branch portal 110b. The circumferentially staggered locations of the openings 119a-119c of the embodiments shown in FIG. 2 can, for example, correspond to locations of the BCA 24, LCCA 25 and LSA 26 in a patient.

The side branch portals 110 may be incorporated into the main body 100 in a variety of ways. For example, the side branch portals 110 may be wrapped between layers of film in the graft member 130. It is noted that in those embodiments in which a plurality of side branch portals 110 are implemented, a plug (not shown) may be inserted into any one or multiple side branch portals 110 if one or more of the side branch portals are not needed in a particular application. For example, a device 10 may include three side branch portals 110, but only two are needed for a patient (e.g., in the aortic arch with a bypass), one of the side branch portals 110 may be closed (e.g., via a plug).

In some embodiments, the stent structure 120 extends around an outer periphery of the side branch portals 110. However, in some embodiments, the stent structure 120 does not extend around the side branch portals 110.

The main body 100 of device 10 is shown in an expanded diameter, unconstrained configuration in FIG. 2, at which it is in an unconstrained state and not subject to external forces that might tend to reduce its diameter. In the illustrated embodiments the main body 100 includes a first portion 201, a second portion 202, and a third portion 203. The first portion 201, which includes the first end 106, has a first unconstrained diameter. The third portion 203, which includes the second end 108, has a third unconstrained diameter. In the illustrated embodiments, the first unconstrained diameter of the first portion 201 and the third unconstrained diameter of the third portion 203 are about the same diameter. However, in other embodiments the first unconstrained diameter of the first portion 201 and the third unconstrained diameter of the third portion 203 may be different (e.g., to accommodate different surgical sites and/or patients with differently-sized anatomical passages).

The second portion 202 of the main body 100, which is located between the first portion 201 and the third portion 203 in the illustrated embodiments, includes one of more of the side branch portals 110 and openings 119. The illustrated embodiments of the second portion 202 includes the three side branch portals 110a, 110b and 110c, for purposes of example. In the illustrated embodiments the second portion 202 has a second unconstrained diameter that is less than the unconstrained diameters of both the first portion 201 and the third portion 203. However, in other embodiments, the unconstrained diameter of the second portion 202 can be equal to or greater than one or both of the unconstrained diameters of the first portion 201 and the third portion 203.

Although the first portion 201, second portion 202 and third portion 203 each have generally constant unconstrained diameters in the illustrated embodiments, in other embodiments the unconstrained diameters of one or more of the first, second or third portions may vary over the lengths of those portions. For example, in embodiments, the unconstrained diameters of one or more of the first portion 201, the second portion 202 or the third portion 203 may have tapered or sloping unconstrained diameters. In general, the unconstrained diameters of the first portion 201, second portion 202 and/or third portion 203 are configured to accommodate the physiology of the particular surgical site and the size of the patient's anatomical passage. The unconstrained diameters of the first portion 201, second portion 202 and/or third portion 203 may also be configured to enable the effective and efficacious delivery and/or deployment of the device 10.

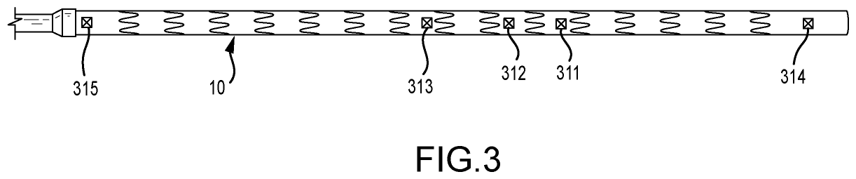

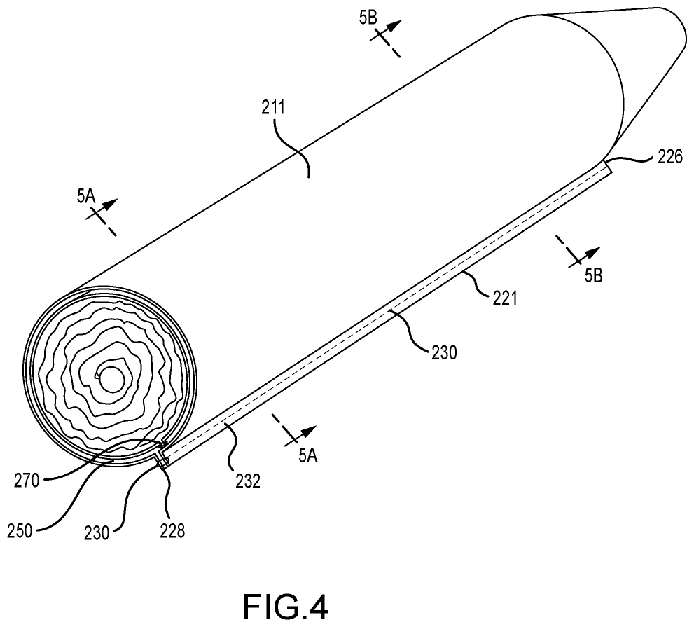

Device 10 is configured for different stages of deployment for implantation in a body of a patient. For example, device 10 includes an undeployed, delivery configuration in which at least a portion of the device is contained in one or more constraints or sleeves that constrain all or portions of the device for delivery into the vasculature of the patient. Device 10 has an undeployed delivery diameter when the device is in the delivery configuration. When in the delivery configuration the device 10 has a reduced diameter configuration with respect to the device in the expanded configuration shown in FIG. 2 to allow delivery of the device to a surgical site in the vasculature of the patient. When the device has been delivered into the vasculature of a patient, the one or more constraints are removed, allowing the device to be deployed and expanded in a manner complementary to the vasculature. For reference, the term “removed” as used with respect to the one or more delivery sleeves is synonymous with “released,” and does not require physical removal from the body. FIGS. 3 and 4, for example, show the device 10 in a delivery configuration.

The one or more delivery sleeves may be comprised of, for example, expanded polytetrafluoroethylene (ePTFE), expanded polyethylene (ePE), polyester, polyurethane, fluoropolymers, such as perfluoroelastomers and the like, polytetrafluoroethylene, silicones, urethanes, ultra-high molecular weight polyethylene, aramid fibers, and combinations thereof. Other instances for the sleeve material may include high strength polymer fibers such as ultra-high molecular weight polyethylene fibers or aramid fibers. The sleeve may include a bioactive agent. Any sleeve which may be used to constrain an endoluminal device is in accordance with the present disclosure.

The illustrated embodiments of the device 10 include a first sleeve 211, a second sleeve 212, and a third sleeve 213. As described in greater detail below, the first sleeve 211 is configured to cover all or portions of the device 10, including all or portions of the first portion 201, second portion 202 and third portion 203 of the body 100, and to constrain the device 10 to the delivery configuration. As noted above, the device 10 is shown in an expanded configuration in FIG. 2, and the first sleeve 211, second sleeve 212 and third sleeve 213 are shown in generally flat or planar forms extending from the main body 100 for purposes of description (e.g., in a released configuration, and not in their constrained or delivery configurations).

The second sleeve 212 is configured to cover at least portions or all of at least the first portion 201 of the body 100 (e.g., in some embodiments it may also cover portions of the second portion 202), and to constrain at least portions or all of at least the first portion 201 of the device 10 at an intermediate configuration during delivery. At the intermediate configuration of the second sleeve 212, portions of the device 10, including at least portions of the first portion 201 of the body 100, have an intermediate diameter that is greater than the delivery diameter and less than the unconstrained diameter, and typically less than the diameter of the first portion when in the deployed configuration.

The third sleeve 213 is configured to cover at least portions or all of at least the third portion 203 of the body 100 (e.g., in some embodiments it may also cover portions of the second portion 202), and to constrain at least portions or all of at least the third portion 203 of the device 10 at an intermediate configuration during delivery. At the intermediate configuration of the third sleeve 213, portions of the device 10, including at least portions of the third portion 203 of the body 100, have an intermediate diameter that is greater than the delivery diameter, less than the unconstrained diameter, and typically less than the diameter of the third portion when in the deployed configuration. At the intermediate configuration of the third sleeve 213, the at least portions of the third portion 203 of the body 100 can have an intermediate diameter that is greater than, generally equal to, or less than the intermediate diameter of the second sleeve 212 when the second sleeve is in its intermediate configuration.

First sleeve 211 includes a first side edge 221, a second side edge 222, a first end edge 223 and a second end edge 224. In the illustrated embodiments, the first sleeve 211 has a length that is sufficient to constrain the first portion 201, second portion 202 and third portion 203 of the body 100 in the delivery configuration. In the illustrated embodiments the first end edge 223 of the first sleeve 211 does not extend to the first end 106 of the body 100. As shown in FIG. 2 for example, at least a portion of a proximal-most one of an undulating length of the stent structure 120, and a portion of the graft member 130 between that proximal-most one of the undulating lengths of the stent structure and the proximal end 106 of the body 100, extend beyond the first end edge 223 of the first sleeve 211. In other embodiments the first end edge 223 of the first sleeve 211 is located closer to (including at) the first end 106 of the main body 100, or further away from the first end of the main body, than the embodiments shown in FIG. 2. In the illustrated embodiments the second end edge 224 of the first sleeve 211 extends to the second end 108 of the main body 100. In other embodiments the second end edge 224 of the first sleeve 211 is spaced apart form second end 108 of the main body 100.

Although the first sleeve 211 is generally rectangular in the illustrated embodiments, the first sleeve can take other shapes in other embodiments. For example, the first end edge 223 and/or the second end edge 224 may include one or more portions that are non-perpendicular with respect to a longitudinal axis of the device 10 that extends between the first end 06 and the second end 108.

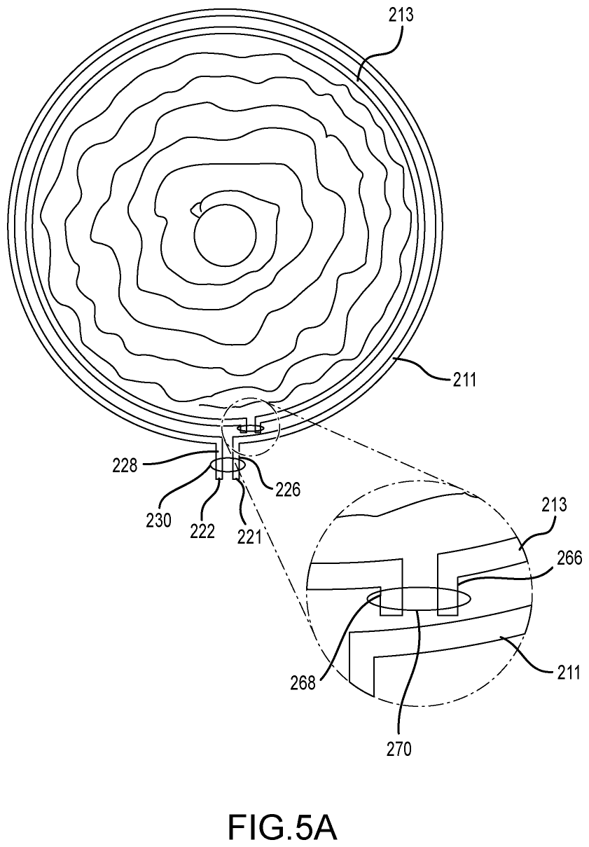

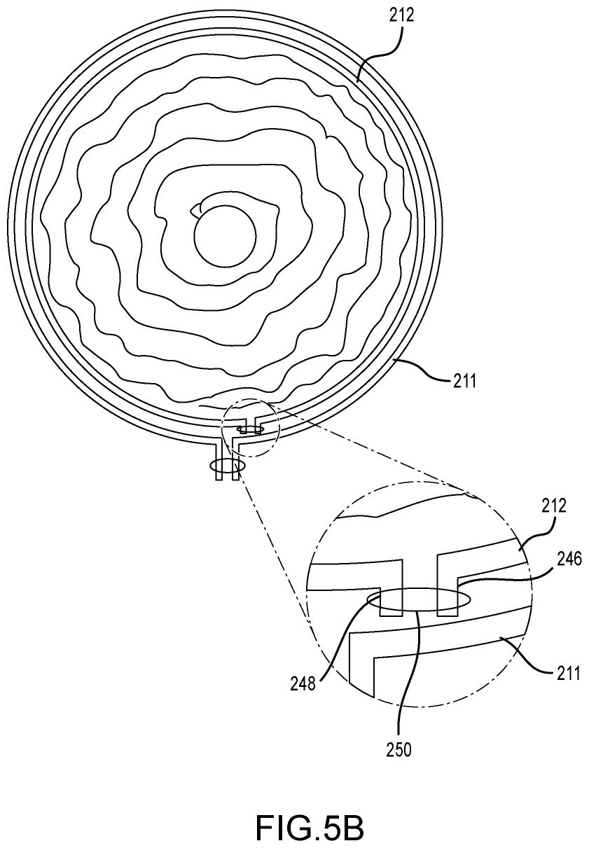

A width of the first sleeve 211 between the first side edge 221 and the second side edge 222 is configured to constrain the first sleeve-covered portions of the body 100 in the delivery configuration when portions of the first sleeve along the first side edge 221 are coupled to portions of the first sleeve along the second side edge 222. FIG. 4, for example, shows the first sleeve 211 in the delivery configuration wrapped around the body 100. As shown in FIGS. 4 and 5A, a first side edge portion 226 of the first sleeve 211 is releasably coupled to a second side edge portion 228 of the first sleeve by a releasable coupler such as a first deployment line 230. Portions of the first deployment line 230 extend between the first end edge 223 and the second end edge 224 of the first sleeve 211, and define a first seamline 232 of the first sleeve 211.

As shown in FIGS. 2 and 4, for example, the first seamline 232 is located about adjacent to the first side edge 221 of the first sleeve 211. In embodiments, for example, the first seamline 232 can be located as close to the first side edge 221 of the first sleeve 211 as possible, while still facilitating the first side edge portion 226 to be and remain coupled to the second side edge portion 228 while the device 10 is being delivered to and positioned at the surgical site in the patient, and to enable the first seamline to be efficiently manufactured. For example, in embodiments the first seamline 232 is located approximately 1-5 mm from the first side edge 221 of the first sleeve. In these or other embodiments, the first seamline 232 may be located from the first side edge 221 by distances referenced with respect to a width of the first sleeve 211, such as for example less than or equal to 2% of the width of the first sleeve, less than or equal to 5% of the width of the first sleeve, or less than or equal to 10% of the first sleeve.

As shown in FIG. 2, the first sleeve 211 is attached to the first body 100 with the first seamline 232 located generally opposite (e.g., diametrically opposite) the body 100 from the openings 119 of the side branch portals 110. For example, in the illustrated embodiments, the first seamline 232 is generally parallel to the longitudinal axis of the main body 100 and located opposite the main body from the third opening 119c of the side branch portal 110c. Locating the first seamline 232 generally opposite the openings 119 of the side branch portals 110 can provide certain advantages in connection with the delivery of the device 10. For example, in surgical applications where the device 10 is being delivered to and located in the aortic arch between the ascending aorta and the descending aorta, the openings 119 may be positioned at the top wall portions of the aortic arch, and the first seamline 232 may be located adjacent the bottom wall portions of the aortic arch. Locating the device 10 with the first seamline 232 adjacent to the bottom wall portions of the aortic arch can enhance the deliverability of the device, for example its ability to bend or curve to the shape of the aortic arch, and the ability of the sleeves such as 201, 202 and/or 203 to release and assume delivery positions between the main body 100 and walls of the aorta, when the sleeves are released and the main body expands to its deployed configurations.

As conventions for purposes of this description, circumferential locations of features of the device 10 can be described with respect to one or both of degrees on a circle or times on a clock face, where 0° and 6:00 define the location of the first seamline 232. Similarly, locations of features of the device 10 with respect to features of the vessels in which the device is being delivered and implanted can be described with reference to degrees on a circle or times on a clock face. For example, in connection with devices 10 implanted in an aorta 20 having branched vessels such as BCA 24, LCCA 25 and LCA 26, 180° and 12:00 can define the locations of the branched vessels, which extend generally from the upper or superior portions of the aortic arch, opposite the lower or inferior portions of the aortic arch that can be defined by 0° and 6:00.

Following these conventions, in the embodiments illustrated in FIG. 2, the 0° and 6:00 locations of the first seamline 232 are locations referenced with respect to the location of opening 119 c that is at 180° and 12:00. In other embodiments, the 0° and 6:00 locations of the first seamline 232 are locations referenced with respect to locations of one of the openings 119a or 119c. In some embodiments, the 0° and 6:00 locations of the first seamline 232 may be locations between 0°-3°, or 6:00-6:06, from a location that is 180° opposite the body 100 from any of the openings 119a-119c. In yet other embodiments, the 0° and 6:00 locations of the first seamline 232 may be locations at or between 3°-10°, or 6:06-6:20, from a location that is 180° opposite the body 100 from any of the openings 119a-119c. In still other embodiments, the 0° and 6:00 locations of the first seamline 232 may be locations at or between 10°-20°, or 6:20-6:40, from a location that is 180° opposite the body 100 from any of the openings 119a-119c. In some embodiments, the 0° and 6:00 locations of the first seamline 232 may be locations at or between 20°-30°, or 6:40-7:00, from a location that is 180° opposite the body 100 from any of the openings 119a-119c. In some embodiments, the 0° and 6:00 locations of the first seamline 232 may be locations at or between 30°-50°, or 7:00-7:40, from a location that is 180° opposite the body 100 from any of the openings 119a-119c. In some embodiments, the 0° and 6:00 locations of the first seamline 232 may be locations at or between 50°-70°, or 7:40-8:20, from a location that is 180° opposite the body 100 from any of the openings 119 a-119c. In yet other embodiments, the 0° and 6:00 locations of the first seamline 232 may be locations greater than 70° or 8:20 from a location that is 180° opposite the body 100 from any of the openings 119a-119c. Although described for purposes of example with respect to positions of the first seamline 232 greater than 6:00 (e.g., in a clockwise direction), it is to be understood that corresponding positions less than 6:00 (e.g., in a counterclockwise direction) may be similarly suitable.

Second sleeve 212 includes a first side edge 241, a second side edge 242, a first end edge 243 and a second end edge 244. In the illustrated embodiments, the second sleeve 212 has a length that is sufficient to constrain at least portions, or all, of the first portion 201 of the main body 100 in its intermediate configuration. In the illustrated embodiments, the first end edge 243 of the second sleeve 212 does not extend to the first end 106 of the body 100. In the illustrated embodiments the first end edge 243 of the second sleeve 212 and the first end edge 223 of the first sleeve 211 are generally flush with the apex tips of a proximal most one of the undulating length of the stent structure 120. In the illustrated embodiments, the first end edge 243 of the second sleeve 212 is generally aligned with the first end edge 223 of the first sleeve 211. In the illustrated embodiments the second end edge 244 of the second sleeve 212 extends to the second portion 202 of the body 100. In other embodiments the second end edge 244 of the second sleeve 212 is spaced apart from the second portion 202 of the main body 100, and does not extend all the way to the second portion of the main body. In embodiments, the second sleeve 212 and the second end edge 244 of the second sleeve are configured such that the second sleeve does not cover any portion of the openings 119, such as the opening 119a of the side branch portal 110a. Although the second sleeve 212 is generally rectangular in the illustrated embodiments, the second sleeve can take other shapes in other embodiments. For example, the first end edge 243 and/or the second end edge 244 may include one or more portions that are non-perpendicular with respect to a longitudinal axis of the main body 100.

A width of the second sleeve 212 between the first side edge 241 and the second side edge 242 is configured to constrain at least the second sleeve-covered portions of the body 100 in the intermediate configuration when the second sleeve is wrapped around the body 100 and portions of the second sleeve along the first side edge 241 are coupled to portions of the second sleeve along the second side edge 242. FIG. 5B, for example, shows the second sleeve 212 in the delivery configuration wrapped around the main body 100. As shown in FIG. 5B, a first side edge portion 246 of the second sleeve 212 is releasably coupled to a second side edge portion 248 of the second sleeve by a releasable coupler such as a second deployment line 250. Portions of the second deployment line 250 extend between the first end edge 243 and the second end edge 244 of the second sleeve 212, and define a second seamline 252 of the second sleeve 212. In embodiments, the second seamline 252 can be located as close to the first side edge 241 of the second sleeve 212, as possible, while enabling the first side edge portion 246 to be and remain coupled to the second side edge portion 248 while the device 10 is being delivered to and positioned at the surgical site in the patient. In these or other embodiments, the second seamline 252 may be located from the first side edge 241 by distances referenced with respect to a width of the second sleeve 212, such as for example less than or equal to 2% of the width of the second sleeve, less than or equal to 5% of the width of the second sleeve, or less than or equal to 10% of the second sleeve. In other embodiments the second seamline 252 is located greater distances from the first side edge 241 of the second sleeve 212.

As shown in FIG. 2, for example, the second sleeve 212 is attached to the main body 100 with the second seamline 252 located about adjacent to the first seamline 232. In embodiments, for example, the second sleeve can be attached to the main body 100 with the second seamline 252 as close to the first seamline 232 as possible, while enabling the first side edge portion 246 to be and remain coupled to the second side edge portion 248 and the body 100 while the device 10 is being delivered to and positioned at the surgical site in the patient, and to enable the attachment to be efficiently manufactured. For example, in some embodiments, the second sleeve 212 is attached to the body 100 with the second seamline 252 is located approximately 1-5 mm from the first seamline 232. In some embodiments, the second sleeve 212 is attached to the body 100 with the second seamline 252 is located approximately 5-10 mm from the first seamline 232. In other embodiments the second sleeve 212 is attached to the main body 100 with the second seamline 252 located greater distances from the first seamline 232.

Third sleeve 213 includes a first side edge 261, a second side edge 262, a first end edge 263 and a second end edge 264. In the illustrated embodiments, the third sleeve 213 has a length that is sufficient to constrain at least portions, or all, of the third portion 203 of the body 100 in its intermediate configuration. In the illustrated embodiments the second end edge 264 of the third sleeve 213 extends to the second end 108 of the body 100. In the illustrated embodiments, the second end edge 264 of the third sleeve 213 is generally aligned with the second end edge 224 of the first sleeve 211. In other embodiments the second end edge 264 of the third sleeve 212 is located further from the second end 108 of the main body 100, or further from the second end edge 224 of the first sleeve 211 than the embodiments shown in FIG. 2. In the illustrated embodiments the first end edge 263 of the third sleeve 213 extends to the second portion 202 of the body 100. In other embodiments the first end edge 263 of the third sleeve 213 is spaced apart from the second portion 202 of the body 100, and does not extend all the way to the second portion of the main body. In embodiments, the third sleeve 213 and the first end edge 263 of the third sleeve are configured such that the third sleeve does not cover any portion of the openings 119, such as the opening 119c of the side branch portal 110c. The first end edge 263 of the third sleeve 213 is generally concave in the illustrated embodiments, and includes portions that are non-perpendicular with respect to the longitudinal axis of the main body 100. Other embodiments of the third sleeve 213 can take other shapes, such as for example rectangular shapes.

A width of the third sleeve 213 between the first side edge 261 and the second side edge 262 is configured to constrain at least the third sleeve-covered portions of the main body 100 in the intermediate configuration when the third sleeve is wrapped around the body 100 and portions of the third sleeve along the first side edge 261 are coupled to portions of the third sleeve along the second side edge 262. FIG. 5A, for example, shows the third sleeve 213 in the delivery configuration wrapped around the main body 100. As shown in FIG. 5A, a first side edge portion 266 of the third sleeve 213 is releasably coupled to a second side edge portion 268 of the third sleeve by a releasable coupler such as a third deployment line 270.

Portions of the third deployment line 270 extend between the first end edge 263 and the second end edge 264 of the third sleeve 213, and define a third seamline 272 of the third sleeve 213. In embodiments, the third seamline 272 can be located as close to the first side edge 261 of the third sleeve 213, as possible, while enabling the first side edge portion 266 to be and remain coupled to the second side edge portion 268 while the device 10 is being delivered to and positioned at the surgical site in the patient, and to enable the third seamline to be efficiently manufactured.

For example, in some embodiments the third seamline 272 is located approximately 1-5 mm from the first side edge 261 of the third sleeve 213. In some embodiments the third seamline 272 is located approximately 5-10 mm from the first side edge 261 of the third sleeve 213. In these or other embodiments, the third seamline 272 may be located from the first side edge 261 by distances referenced with respect to a width of the third sleeve 213, such as for example less than or equal to 2% of the width of the third sleeve, less than or equal to 5% of the width of the third sleeve, or less than or equal to 10% of the third sleeve. In other embodiments the third seamline 272 is located greater distances from the first side edge 261 of the third sleeve 213.

As shown in FIG. 2, for example, the third sleeve 213 is attached to the body 100 with the third seamline 272 located about adjacent to the first seamline 232. In embodiments, for example, the third sleeve 213 can be attached to the main body 100 with the third seamline 272 as close to the first seamline 232 as possible, while enabling the first side edge portion 266 to be and remain coupled to the second side edge portion 268 and the main body 100 while the device 10 is being delivered to and positioned at the surgical site in the patient, and to enable the attachment to be efficiently manufactured. For example, in some embodiments third sleeve 212 is attached to the body 100 with the third seamline 272 located approximately 1-5 mm from the first seamline 232. In some embodiments third sleeve 212 is attached to the body 100 with the third seamline 272 located approximately 5-10 mm from the first seamline 232. In other embodiments the third sleeve 213 is attached to the main body 100 with the third seamline 272 located greater distances from the first seamline 232.

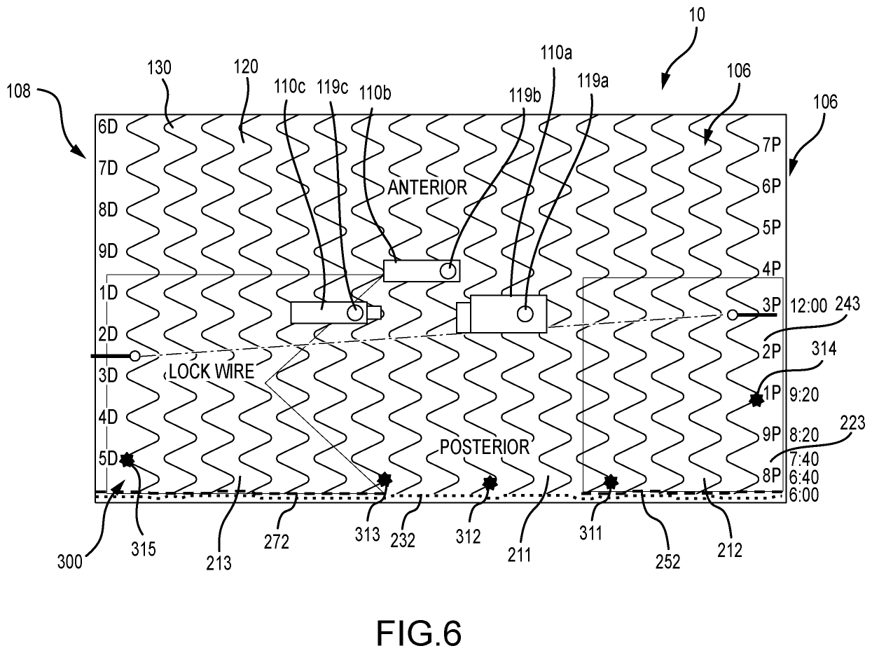

FIG. 6 is an illustration of an embodiment of the device 10 such as that shown in FIG. 2 in a flat or unrolled form corresponding to its expanded configuration, rather than the cylindrical or tubular form show in FIG. 2, for purposes of description of certain features of the device. For example, attachment locations 300 at which the first sleeve 211, second sleeve 212 and third sleeve 213 are attached to the body 100 in accordance with certain embodiments can be described with reference to FIG. 6. In embodiments, the attachments at the attachment locations 300 are sutures that tie the first sleeve 211, second sleeve 212 and/or third sleeve 213 to the main body 100 of the device 10.

In FIG. 6, apices of the undulating members of the stent structure 120 adjacent the proximal or first end 106 of the main body 100 are labeled 1P-9P.

Apices of the undulating members of the stent structure 120 adjacent the distal or second end 108 of the body 100 are labeled 1D-9D. First sleeve 211, second sleeve 212 and third sleeve 213, and the locations of their associated seamlines 232, 252 and 272, respectively, shown overlaid on the body 100. The side branch portals 110a-110c are also shown on the body 100. Consistent with the conventions used above, circumferential locations of certain features of the device 10 are identified by degrees on a circle or times on a clock face. Consistent with the descriptions of device 10 above, the location of the first seamline 232 is shown at 0° and 6:00 on the body 100, and the locations of the openings 119a-119c are shown generally at 180° and 12:00.

The first sleeve 211 is attached to the body 100 at attachment locations 300 that are circumferentially located sufficiently close to the first seamline 232 to prevent the first sleeve from substantially covering any of the openings 119 of the side branch portals 110 after the first deployment line 230 is actuated to release the first edge portion 226 from the second edge portion 228, and the second portion 202 of the device is expanded from its constrained configuration to its deployed configuration when the device is located in the vessel of the patient. In embodiments, the first sleeve 211 is attached to the body 100 at attachment locations 300 that are circumferentially located sufficiently close to the first seamline 232 to prevent the first sleeve from substantially covering any of the openings 119 of the side branch portals 110 after the second sleeve 212 expands to its intermediate configuration following the release of the first sleeve 211, and/or after the second deployment line 250 is actuated to release the first edge portion 246 from the second edge portion 248 and the first portion 201 is expanded to its deployed configuration when the device 10 is located in the vessel of the patient. In embodiments, the first sleeve 211 is attached to the body 100 at attachment locations 300 that are circumferentially located sufficiently close to the first seamline 232 to prevent the first sleeve from substantially covering any of the openings 119 of the side branch portals 110 after the third sleeve 213 expands to its intermediate configuration following the release of the first sleeve 211, and/or after the third deployment line 270 is actuated to release the first edge portion 266 from the second edge portion 268 and the third portion 203 is expanded to its deployed configuration when the device 10 is located in the vessel of the patient.

As used herein, the term “substantially covering” means an amount of covering that results in the associated opening 119 being covered or occluded by an amount that would, for example, either prevent the device 10 from being deployed in the patient, and/or cause the opening to be covered or occluded by an amount that would prevent the deployed device from functioning in an efficacious manner following deployment in the patient. For example if one or more of the first sleeve 211, second sleeve 212 or third sleeve 213 covers one or more of the openings 119 by an amount that cannulation of a side branch vessel such as the BCA 24, LCCA 25 or LCA 26, cannot be performed, or a branch body 300 cannot be deployed, the sleeve may be considered to be substantially covering the one or more openings.

As another example, if one or more of the first sleeve 211, second sleeve 212 or third sleeve 213 covers or occludes one or more of the openings 119 by an amount that prevents sufficient blood flow through the opening to provide medically efficacious treatment, the sleeve may be considered to be substantially covering the one or more openings. As used herein, terms such as “to prevent” “substantially covering” also mean the absence of any covering, for example no covering The second sleeve 212 is attached to the main body 100 at attachment locations 300 that are circumferentially located sufficiently close to the first seamline 232 to prevent the second sleeve from substantially covering any of the openings 119 of the side branch portals 110 after the second deployment line 250 is actuated to release the first edge portion 246 from the second edge portion 248, and the first portion 201 of the device is expanded from its intermediate configuration to its deployed configuration when the device is located in the vessel of the patient.

In embodiments that include a third portion 203 and a third sleeve 213, the third sleeve is attached to the main body 100 at attachment locations 300 that are circumferentially located sufficiently close to the first seamline 232 to prevent the third sleeve from substantially covering any of the openings 119 of the side branch portals 110 after the third deployment line 270 is actuated to release the first edge portion 266 from the second edge portion 268, and the third portion 203 of the device is expanded from its intermediate configuration to its deployed configuration when the device is located in the vessel of the patient.

In some embodiments, the first sleeve 201 is attached to the main body 100 at plurality of attachment locations 300, such as first attachment locations 311, 312 and 313, that are located within about 30° of the first seamline 232 (e.g., at or between 6:00 and 7:00). In some embodiments, the first attachment locations 311, 312 and 313 are located within about 15° of the first seamline 232 (e.g., at or between 6:00 and 6:30). In the illustrated embodiments, the first attachment locations 311, 312 and 313 are located at about 7° from the first seamline 232. In the illustrated embodiments, the first attachment location 312 is a location on only the first sleeve 211, between the second end edge 244 of the second sleeve 212 and the first end edge 263 of the third sleeve 213. In the illustrated embodiments the first attachment location 312 is at a location generally centered between the second end edge 244 of the second sleeve 212 and the first end edge 263 of the third sleeve 213. The first attachment location 311 is a location on the first sleeve 211 that overlies a portion of the second sleeve 212, between the first attachment location 312 and the first end 106 of the main body 100. As described below, in embodiments the first attachment location 311 also attaches the second sleeve 212 to the main body 100. In the illustrated embodiments the first attachment location 311 is located closer to the second end edge 244 of the second sleeve 212 than to the first end edge 243 of the second sleeve. The first attachment location 313 is a location on the first sleeve 211 that overlies a portion of the third sleeve 213, between the first attachment location 312 and the second end 108 of the main body 100. As described below, in embodiments the first attachment location 313 also attaches the third sleeve 213 to the main body 100. In the illustrated embodiments the first attachment location 313 is located closer to the first end edge 263 of the third sleeve 213 than to the second end edge 264 of the third sleeve.

In the embodiments described above, the first attachment locations 300 include at least one attachment location such as 311 and 312 on each of the first portion 201 and the second portion 202, respectively, of the main body 100. In the illustrated embodiments the first attachment locations 300 include at least one attachment location such as 311 on the first portion 201 of the main body 100, and at least two locations such as 312 and 313 on the second portion 202 of the main body 100. Other embodiments of the device 10 include more or fewer that the three first attachment locations 311, 312 and 313 shown in FIG. 6, and or first attachment locations at other locations between the first end 106 and the second end 108 of the body 100.

In the illustrated embodiments, the attachment locations 300 include one or more attachment locations that attach the first sleeve 211 and the second sleeve 212 sufficiently close to the first seamline 232 to prevent the first sleeve and the second sleeve from overhanging the opening 107 one the first end 106 of the main body 100, and/or from substantially covering the opening 107 when the first portion 201 and the second portion 202 of the body have expanded to their deployed configurations. For example, in the illustrated embodiments the attachment locations 300 include at least one second attachment location 314 between the first attachment locations 311, 312 and 313 and the first end 106 of the device 10. In embodiments, each of the second attachment locations such as 314 is a location at or between about 30° and about 65° from the first seamline 232. In some embodiments, each of the second attachment locations such as 314 is a location at or between about 50° and about 60° from the first seamline 232. In the illustrated embodiment, the second attachment location 314 is at about 55° or 9:20 from the first seamline 232. In the illustrated embodiment the second attachment location 314 is about adjacent to the first end edge 223 of the first sleeve 211 and attaches the first sleeve to the body 100. In the illustrated embodiment the second attachment location 314 is adjacent to the first end edge 243 of the second sleeve 212 and attaches the second sleeve to the main body 100. The second attachment location 314 attaches both the first sleeve 211 and the second sleeve 212 to the body 100 in embodiments.

In the illustrated embodiments, the attachment locations 300 include one or more attachment locations that attach the first sleeve 211 and the third sleeve 213 sufficiently close to the first seamline 232 to prevent the first sleeve and the third sleeve from overhanging the opening 109 at the second end 108 of the main body 100, and/or from substantially covering the opening 109, when the first portion 201 and the third portion 203 of the main body have expanded to their deployed configurations. For example, in the illustrated embodiments the attachment locations 300 include at least one third attachment location 315 between the first attachment locations 311, 312 and 313 and the second end 108 of the device 10. In embodiments, each of the third attachment locations such as 315 is a location at or between about 30° and about 50° from the first seamline 232. In some embodiments, each of the third attachment locations such as 315 is a location at or between about 35° and about 45° from the first seamline 232. In the illustrated embodiment, the third attachment location 315 is at about 40° or 7:20 from the first seamline 232. In the illustrated embodiment the third attachment location 315 is about adjacent to the second end edge 224 of the first sleeve 211 and attaches the first sleeve to the main body 100. In the illustrated embodiment the third attachment location 315 is about adjacent to the second end edge 264 of the third sleeve 213 and attaches the third sleeve to the main body 100. The third attachment location 315 attaches both the first sleeve 211 and the third sleeve 213 to the main body 100 in embodiments.

Branched endoluminal devices such as 10 and associated branch bodies such as 30 (e.g., FIG. 1) can be implanted in vessels of patients to treat indications such as disease or other conditions. For example, the embodiments of the device 10 can be implanted into the aorta of a patient, and one or more of the side branch portals 110 can be coupled to one or more of the patient's branch vessels such as BCA 24, LCCA 25 and/or LCA 26 by a branch body such as 30a, 30b and/or 30c, respectively, shown in FIG. 1.

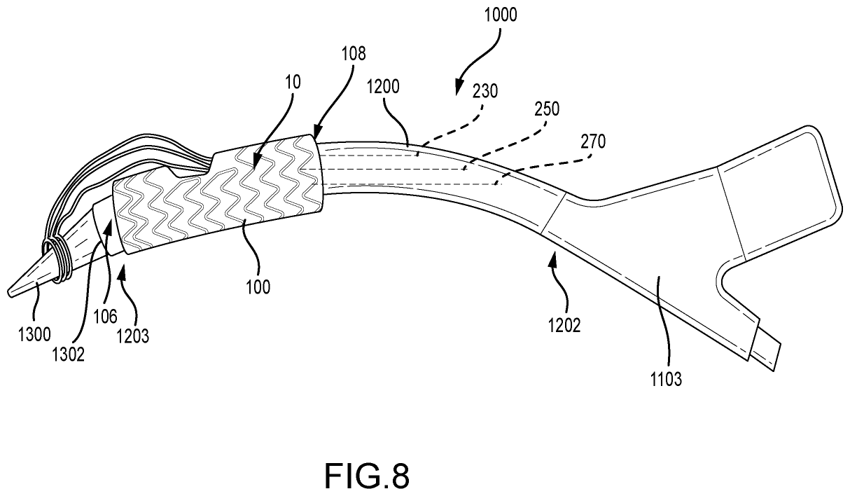

FIG. 8 illustrates a delivery system 1000 (not necessarily to scale) that can be used to implant the device 10. The delivery system 1000 is operable to deliver a multi-component implantable device (e.g., implantable device 10) to a target site. The delivery system includes a handle 1100, an elongate member 1200 having a first end 1202 coupled to and/or extending from the handle 1100 and a second end 1204, a cap 1300 positioned proximate the second end 1204 of the elongate member 1200, and at least one guide member 1400 extending at least partially along the elongate member 1200 toward the cap 1300. The elongate member 1200 and cap 1300 may be operable to translate along a main guidewire.

In the illustrated embodiments, the leading or proximal end 106 of the device 10 is located on a distal portion of the elongate member 1200. The first, second and third deployment lines 230, 250, 270 extend from the device 10 toward the handle 1100. The delivery system 1000, and the operation of the delivery system to deliver, locate and deploy (e.g., to implant) the device 10, and to deliver, locate and deploy (e.g., to implant) branch bodies such as 30, can for example be the same as or similar to those described in the Birdno U.S. Patent Application Publication 2024/0130877.

During the methods of treatment, the delivery system 1000 can be manipulated to advance the device 10 thereon, when in its delivery state, to the aorta, for example through the descending aorta. By this method the device 10 is advanced through and located in the aortic arch. During this advancement of the device 10, and/or after the device is located in the aortic arch, the side of the device 10 including one or more of the side branch portals 110 is located generally at the 180° or 12:00 location of the aortic arch (e.g., the 12:00 position of the device 10 is located at the 12:00 or superior side of the aortic arch, generally facing the branching arteries). For example, in some embodiments, the handle 1100 can be used to rotate the device 10 during the advancement of the device 10 through the descending aorta and/or the aortic arch. The device 10 is thereby initially positioned or located in the aortic arch, with the side branch portals 110 of the device oriented toward or facing the branching arteries, and the first, second and third seamlines 232, 252 and 272 oriented toward or facing the inferior side of the aorta (e.g., the 0° or 6:00 location of the aortic arch). Following this initial positioning of the device 10, the one or more openings 119 of the side branch portals 110 are at least initially positioned with respect the associated one or more branching arteries.

After the deployment system 10 is at least initially positioned, the device 10 is actuated to release the first seamline 232 (e.g., while the first seamline is located adjacent to the inferior side of the aortic arch). The first and second side edge portions 221, 222 of the first sleeve 211 can then separate, releasing the first sleeve 211 and (1) causing the second portion 202 of the device 10 to expand to its deployed configuration, thereby uncovering the openings 119, (2) causing the first portion 201 of the device to expand to its intermediate configuration, and (3) causing the third portion 203 of the device to expand to its intermediate configuration.

Because of the at least initial positioning of the device 10 before the first sleeve 211 is released, the openings 119 of the side branch portals 110 may be positioned relatively close to the one or more associated branch arteries to which they are to be coupled. Because of the above-described features of the device 10, the first sleeve 211 will not substantially cover any of the openings 119 and/or not substantially cover either or both of the openings 107 or 109 of the main lumen 102. And because the first portion 201 and third portion 203 of the device 10 are in their intermediate configurations that may have diameters less than the diameter of the aortic arch or other portions of the vessel in which they are located, the device 10 may optionally be relatively efficiently repositioned, if desired, to better align one or more of the openings 119 with the associated branch vessels.

Following the release of the first sleeve 211 and the expansion of the second portion 202 to its deployed configuration, the device 10 can be actuated to release one or both of the second seamline 250 or the third seamline 270, and to cause the associated first and side edge portions of the associated second sleeve 212 and/or third sleeve 213 to separate, releasing the associated sleeve and causing the associated first portion 201 and/or third portion 203 to expand from its intermediate configuration to its deployed configuration. In its deployed configuration that one of the first portion 201 or the third portion 203 can engage the aorta.

Because of the above-described features of the device 10, the released second or third sleeve will not substantially cover any of the openings 119 and/or not substantially cover either or both of the openings 107 or 109 of the main lumen 102.

Also following the release of the first sleeve 211 and the expansion of the second portion 202 to its deployed configuration, the device 10 can be actuated to release the other one of the second seamline 250 or the third seamline 270, and to cause the associated first and side edge portions of the other associated second sleeve 212 or third sleeve 213 to separate, releasing the associated sleeve and causing the associated first portion 201 or third portion 203 of the device to expand from its intermediate configuration to its deployed configuration. In its deployed configuration that one of the first portion 201 or the third portion 203 can engage the aorta. Because of the above-described features of the device 10, the released other of the second sleeve 212 or third sleeve 213 will not substantially cover any of the openings 119 and/or substantially cover either or both of the openings 107 or 109 of the main lumen 102.

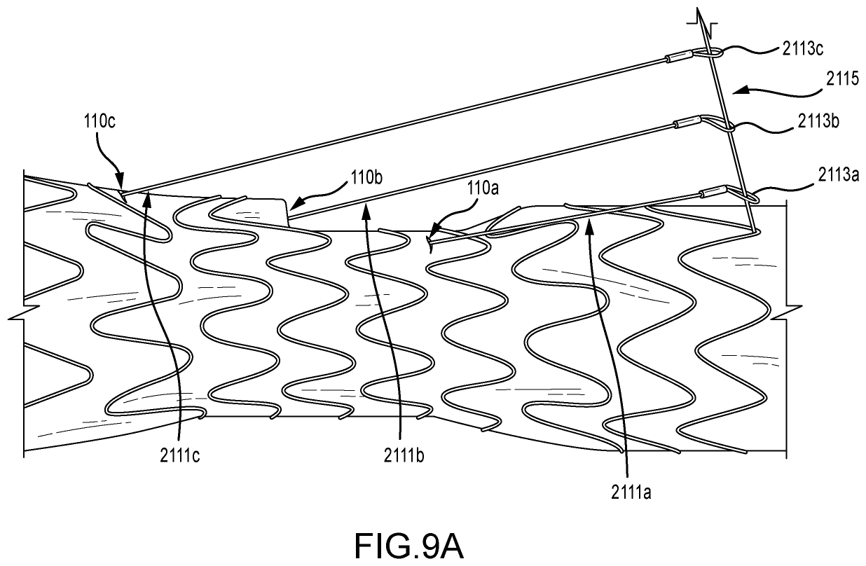

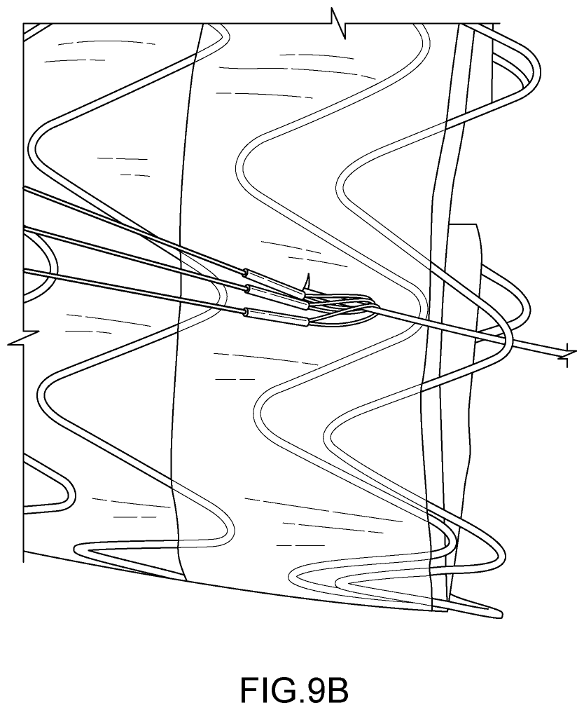

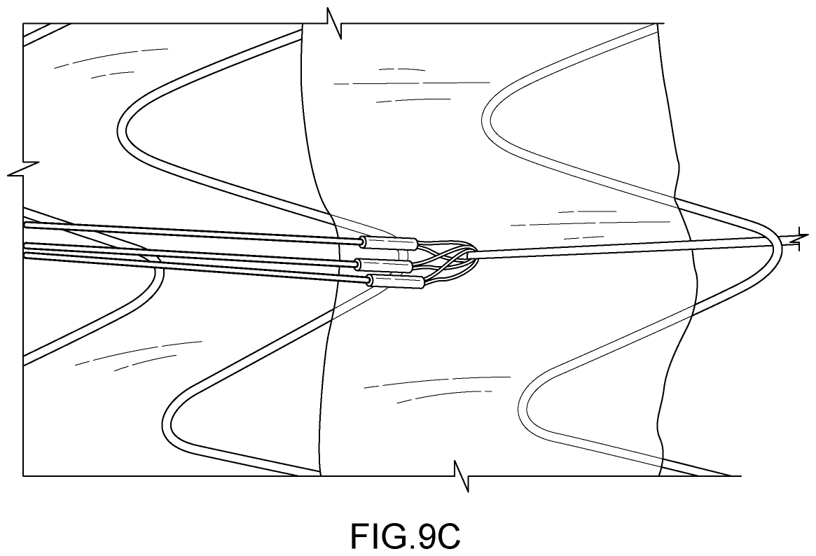

FIGS. 9A-9C illustrate portions of a delivery system 2000 that can be used to implant the device 10, in accordance with embodiments. As shown, guide rails 2111a, 2111b and 2111c extend from the first side branch portal 110a, second side branch portal 110b and third side branch portal 110c, respectively. Guide rails 2111a-2111c have structures such as loops 2113a-2113c on their ends that are configured to receive a lockwire 2115. As shown in FIGS. 9B and 9C, the lockwire 2115 can extend from the loops 2113a-2113c through an apex of the stent structure 120, shown for example on an end of the device 10. In one embodiment the lockwire, or lockwires may extend through a portal(s) and further may extend through a portion of the stent graft (e.g., stent apex, graft material, etc.) and be configured to be releasably attached to the olive (e.g., around the cap 1300 as shown in FIG. 8, into the cap 1300 for example into a hole bore in the cap 1300, etc.).

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A deployment system for a branched endoluminal device, comprising:
an expandable endoluminal device having an exterior surface and an interior surface defining a sidewall, and including at least a first portion and a second portion expandable from a collapsed delivery diameter configuration toward deployed diameter configurations, wherein the second portion includes one or more openings through the sidewall;
a first sleeve including first and second edge portions, the first sleeve having a delivery configuration circumferentially wrapped around at least portions of the first and second portions of the endoluminal device with the first and second edge portions releasably coupled to define a first sleeve seamline substantially opposite the one or more openings, and wherein the first sleeve is attached to the endoluminal device at a plurality of attachment locations circumferentially located sufficiently close to the first sleeve seamline to prevent the first sleeve from substantially covering any of the one or more openings after the first and second edge portions are released and the first and second portions of the endoluminal device have expanded to the deployed diameter configurations; and
a second sleeve including first and second edge portions, the second sleeve having a delivery configuration circumferentially wrapped around the first portion of the endoluminal device with the first and second edge portions releasably coupled to define a second sleeve seamline adjacent to the first sleeve seamline, and wherein the second sleeve is attached to the endoluminal device at a plurality of attachment locations circumferentially located sufficiently close to the first seamline to prevent the second sleeve from substantially covering any of the one or more openings after the first and second edge portions are released and the second portion of the endoluminal device has expanded to the deployed diameter configuration.

2. The deployment system of claim 1, wherein the first sleeve is attached to the endoluminal device at attachment locations including first locations within about 30° from the first sleeve seamline.

3. The deployment system of claim 1, wherein the first sleeve is attached to the endoluminal device at attachment locations including first locations within about 15° from the first sleeve seamline.

4. The deployment system of claim 1, wherein the first locations include at least one location on each of the first portion and the second portion of the endoluminal device.

5. The deployment system of claim 4, wherein the first locations include at least one location on the first portion of the endoluminal device, and at least two locations on the second portion of the endoluminal device.

6. The deployment system of claim 5, wherein at least one of the first locations is a location attaching both the first sleeve and the second sleeve to the endoluminal device.

7. The deployment system of claim 5, wherein:
the endoluminal device includes a proximal end on the first portion, opposite the first portion from the second portion; and
the first sleeve and the second sleeve are attached to the endoluminal device sufficiently close to the first sleeve seamline to prevent the first sleeve and the second sleeve from substantially covering the proximal end of the endoluminal device after the first and second portions of the endoluminal device have expanded to the deployed diameter.

8. The deployment system of claim 7, wherein the first sleeve and the second sleeve are attached to the endoluminal device at attachment locations including second locations between the first locations and the proximal end of the endoluminal device, and wherein the second locations are locations between about 30° and about 65° from the first seamline.

9. The deployment system of claim 8, wherein the second locations are locations between about 50° and about 60° from the first seamline.

10. The deployment system of claim 8, wherein the second locations are on a proximal end portion of the endoluminal device.

11. The deployment system of claim 8, wherein the second locations are the same locations.

12. The deployment system of claim 1, wherein:
the endoluminal device includes a third portion opposite the second portion from the first portion, and wherein the third portion is expandable from a collapsed delivery diameter configuration toward a deployed diameter configuration;
the first sleeve, in the delivery configuration, circumferentially wraps around at least a portion of the third portion of the endoluminal device, and wherein the first sleeve is attached to the endoluminal device at a plurality of attachment locations circumferentially located sufficiently close to the first sleeve seamline to prevent the first sleeve from substantially covering any of the one or more openings after the first, second and third portions of the endoluminal device have expanded to the deployed diameter configurations;
the deployment system comprises a third sleeve including first and second edge portions, the third sleeve having a delivery configuration circumferentially wrapped around the third portion of the endoluminal device with the first and second edge portions releasably coupled to device to define a third seamline, and wherein the third sleeve is attached to the endoluminal device at a plurality of attachment locations circumferentially located sufficiently close to the first seamline to prevent the third sleeve from substantially covering any of the one or more openings after the first and second edge portions are released and the third portion of the endoluminal device has expanded to the deployed diameter configuration.

13. The deployment system of claim 12, wherein the first sleeve is attached to the endoluminal device at attachment locations including first locations within about 30° from the first sleeve seamline.

14. The deployment system of claim 12, wherein the first locations include at least one location on each of the second portion and the third portion of the endoluminal device.

15. The deployment system of claim 14, wherein at least one of the first locations is a location attaching both the first sleeve and the third sleeve to the endoluminal device.

16. The deployment system of claim 14, wherein:
the endoluminal device includes a distal end on the third portion, opposite the third portion from the second portion;
the first sleeve and the third sleeve are attached to the endoluminal device sufficiently close to the first sleeve seamline to prevent the first sleeve and the third sleeve from substantially covering the distal end of the endoluminal device after the first and third portions of the endoluminal device have expanded to the deployed diameter.

17. The deployment system of claim 16, wherein the first sleeve and the third sleeve are attached to the endoluminal device at attachment locations including second locations between the first locations and the distal end of the endoluminal device, and wherein the second locations are locations between about 30° and about 50° from the first seamline.

18. The deployment system of claim 17, wherein the second locations are locations between about 35° and about 45° from the first seamline.

19. The deployment system of claim 17, wherein the second locations are on a distal end portion of the endoluminal device.

20. The deployment system of claim 17, wherein the second locations are the same locations.

21. A method for treating vessel of a patient including one or more branches with a deployment system including a branched endoluminal device, wherein the endoluminal device includes a first portion that is expandable to a first portion deployed configuration, and at least a second portion including one or more side branch openings that is expandable to a second portion deployed configuration, comprising:
delivering the deployment system to the branched vessel while the first and second portions of the endoluminal device are constrained by a first sleeve to a delivery configuration and the first portion is constrained by a second sleeve to an intermediate deployed configuration, and positioning the deployment system with the one or more side branch openings of the endoluminal device facing the one or more branches of the vessel;
actuating the deployment system to release the first sleeve, to cause the second portion of the endoluminal device to expand to its second portion deployed configuration, and to cause the first portion of the endoluminal device to expand to its intermediate deployed configuration, wherein following the release of the first sleeve the first sleeve does not substantially cover any of the side branch openings of the endoluminal device; and
after actuating the deployment system to release the first sleeve, actuating the deployment system to release the second sleeve, to cause the first portion of the endoluminal device to expand to its first portion deployed configuration, wherein following the release of the second sleeve the second sleeve does not substantially cover any of the side branch openings of the endoluminal device.

22. The method of claim 21, wherein the endoluminal device includes a third portion opposite the second portion from the first portion and that is expandable to a third portion deployed configuration, and wherein:
delivering and positioning the deployment system while the first and second portions of the endoluminal device are constrained by the first sleeve to the delivery configuration comprises delivering the deployment system to the branched vessel while the third portion is constrained by the first sleeve to the delivery configuration and the third portion is constrained by a third sleeve to an intermediate deployed configuration;
actuating the deployment system to release the first sleeve comprises causing the third portion of the endoluminal device to expand to its intermediate deployed configuration; and
after actuating the deployment system to release the first sleeve, actuating the deployment system to release the third sleeve, to cause the third portion of the endoluminal device to expand to its third portion deployed configuration, wherein following the release of the third sleeve the third sleeve does not substantially cover any of the side branch openings of the endoluminal device.

23. The method of claim 21, comprising repositioning the deployment system after actuating the deployment system to release the first sleeve, and before actuating the deployment system to release one or both of the first sleeve or the second sleeve.
